(19) United States
(12) Patent Application Publication
(10) Pub. No.: US 2026/0240875 A1
(43) Pub. Date: Aug. 20, 2026

(54) THE USE OF CANNABIDIOL (CBD) AS A COMBINATION TREATMENT FOR GLIOBLASTOMA

(71) Applicant: UNIVERSITY OF SOUTH FLORIDA, Tampa, FL (US)

(72) Inventors: Subhra Mohapatra, Tampa, FL (US); Shyam S. Mohapatra, Tampa, FL (US); Ryan Green, Tampa, FL (US); Karthick Mayilsamy, Tampa, FL (US)

(21) Appl. No.: 19/541,961

(22) Filed: Feb. 17, 2026

Related U.S. Application Data
(60) Provisional application No. 63/758,729, filed on Feb. 14, 2025.
(60) Provisional application No. 63/789,235, filed on Apr. 15, 2025.

(51) Int. Cl. A61K 31/00; A61K 9/107; A61K 31/495; A61K 31/7105; A61K 31/711; A61K 38/17; A61K 38/19; A61P 35/00

(52) U.S. Cl. CPC A61K 31/658; A61K 9/1075; A61K 31/495; A61K 31/7105; A61K 31/711; A61K 38/1793; A61K 38/195; A61P 35/00

(57) ABSTRACT

Glioblastoma is an extremely rare form of brain cancer that has a very low survival rate. Cannabinoid compounds such as cannabidiol (CBD) have been shown to have anti-cancer effects in glioblastoma, which can synergize with glioblastoma therapeutics such as temozolomide (TMZ). However, mono- or combination therapies of CBD can inadvertently activate resistance mechanisms, such as through increased expression or activation of the C-C motif chemokine ligand 20 (CCL20) and its receptor (CCR6) axis. Furthermore, while CBD has been shown to synergize with TMZ to enhance potency against glioblastoma, the heterogeneity of glioblastoma within tumors and amongst patients is challenging to treat, necessitating more personalized combination therapies. Disclosed herein are compositions and methods for treating cannabidiol (CBD)- and/or temozolomide (TMZ)-refractory glioblastoma.

### CROSS-REFERENCE TO RELATED APPLICATIONS

This application claims the benefit of priority to U.S. Provisional Application 63/758,729, filed Feb. 14, 2025, and U.S. Provisional Application 63/789,235, filed Apr. 15, 2025, the disclosures of which are incorporated by reference herein in their entireties.

### TECHNICAL FIELD

This disclosure relates to glioblastoma, and more particularly to refractory glioblastoma.

### BACKGROUND

Glioblastoma, or interchangeably glioblastoma multiforme (GBM), is the most common and deadly brain tumor, with a low 5-year survival rate due to its resistance to chemotherapy, radiation, and immune therapies, as well as the difficulty of surgical resection in the brain. The heterogeneity of GBM, both between patients and within tumors, complicates the selection of effective treatments to overcome drug resistance. Temozolomide (TMZ) is a standard-of-care treatment for GBM that induces DNA damage and cell cycle arrest via methylation of guanine. However, resistance to TMZ often arises due to the activation of DNA repair mechanisms (such as O-6-methylguanine-DNA methyltransferase), pro-survival signaling pathways, autophagy, cell cycle checkpoint defects, and microenvironmental-modulating effects.

Cannabidiol (CBD) has been found to synergize with TMZ in GBM through multiple mechanisms involving TRPV1/4, CB1/CB2, and increased reactive oxygen species (ROS), reducing the viability of GBM cell lines and the growth of syngeneic GBM tumors. However, resistance to this combination therapy remains a concern. Preliminary data indicate that CBD mono or combination therapy can increase the expression/activation of the CC chemokine ligand 20 (CCL20) and its receptor (CCR6) axis in GBM tumor models. CCR6 activation has been linked to tumor survival signaling, progression, metastasis, and poor clinical outcomes in GBM. What are thus needed are new therapies for treating GBM. The compositions and methods disclosed herein address these and other needs.

### SUMMARY

Disclosed herein are compositions and methods of using said compositions. For example, disclosed herein are compositions for treating CBD- and/or TMZ-refractory glioblastoma comprising a cannabinoid, temozolomide (TMZ), a CCL20/CCR6 inhibitor, or a pharmaceutically acceptable salt thereof. In other examples, disclosed herein are compositions for treating CBD- and/or TMZ-refractory glioblastoma comprising a cannabinoid, temozolomide (TMZ), and a polyunsaturated fatty acid. Methods of treating CBD- and/or TMZ-refractory glioblastoma comprising administering a therapeutically effective amount of a cannabinoid, TMZ, a CCL20/CCR6 inhibitor, or pharmaceutically acceptable salts thereof are also disclosed. Further, methods of treating CBD- and/or TMZ-refractory glioblastoma comprising administering a therapeutically effective amount of a cannabinoid, TMZ, and a polyunsaturated fatty acid, are also disclosed.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

FIGS. 1A-1D provide graphs demonstrating that CBD+TMZ treatment show potential for synergy in GBM but individually, the drugs can activate resistance pathways including CCR6. (FIG. 1A) CT-2A, U87, and U251 cells were treated with varying concentrations of CBD, TMZ, or combination and viability were assayed after 48 hr using CellTiterGlo (Promega). All cell lines were more sensitive to CBD than TMZ with CBD IC₅₀s of 35, 80, and 100 μM and TMZ IC₅₀s ranging from 2-3 mM. The addition of 500 μM TMZ to CBD treatment synergistically reduced the CBD IC₅₀ from 35-25 μM in CT-2A. However, CBD+TMZ combinations were antagonistic in U87 and U251. Gene expression was analyzed after 48 hr treatment using RT-qPCR. (FIGS. 1B-1C) TMZ treatment resulted in elevated expression of CCL20 and the HSP70 family member HSPA6 in both U251 and U87. These increases were recovered toward baseline by the addition of CBD. However, CBD alone was observed to increase the expression of CCR6 in U251. (FIG. 1D) In CT-2A, TMZ treatment resulted in increased expression of both CCL20 and CCR6, which were recovered to baseline with the addition of CBD. However, TMZ treatment also elicited upregulation of the pro-inflammatory cytokine IL-6 and the serine protease Upa, which is associated with increased tumor growth and invasion. Both continued to be upregulated in the CBD+TMZ group. ANOVA (Dunnett) n=3*=P≤0.05, **=P≤0.01, ***=P≤0.001.

FIGS. 2A-2B provide graphs demonstrating that CCR6/CCL20 knockdown improves CBD+TMZ therapy in vivo. C56 BL/6 mice (n=6) were injected subcutaneously on the right hind flank with 5×10⁵ CT-2A GBM cells and tumor growth was monitored using a vernier caliper. When tumors reached a size of 6 mm in diameter (day 16), mice were treated with control, CBD (25 mg/kg), temozolomide (20 mg/kg), or the combination via the IP route every alternate day. Control and combination groups (n=10) were split in two with mice either receiving weekly injections of PAMAM dendrimer carrying CCR6+CCL20 shDNA or scrambled control plasmids. At the experiment endpoint (D35), CBD, TMZ, and combination groups had significantly reduced tumor growth compared to control, but none of these groups were different from each other. (FIG. 2A) However, the ShCombo plasmid+CBD+TMZ group had significantly reduced tumor growth compared to all other groups. There was no significant difference in body weight between groups, and all groups gained or maintained body weight over the course of the experiment. *=compared to control, #=compared to TMZ+CBD ANOVA with Holm Sidack p<0.05. (FIG. 2B) Tumors were excised and weighed, and only the triple combo group was observed to have a significantly reduced tumor weight compared to the control. ANOVA (Dunnett) *=P≤0.05.

FIG. 3 provides graphs demonstrating that CBD+TMZ+ShCCR6/CCL20 alters the abundance of lymphocyte and monocyte populations in the spleen of tumor-bearing mice. Spleens were collected from CT-2A flank tumor-bearing mice, and splenocytes were subjected to immunophenotyping by flow cytometry. All treatments significantly increased the abundance of CD3+CD8+ T cells in the spleen, and TMZ, TMZ+CBD, ShCombo, and triple combo increased the proportion of CD3+CD8+ T cells in the spleen. Furthermore, ShCombo and triple combo treatments significantly decreased the frequency of Ly6g Ly6c^{hi} monocytes, while triple combo also increased the proportion of the CD11b+Ly6g+ population, which includes neutrophils. n=5*=compared to control, #=compared to TMZ, ANOVA with Holm Sidack p<0.05

FIG. 4 provides graphs demonstrating qPCR for the expression of gene targets under inflammatory, apoptotic, and other cellular pathways. Treatments were given to U251, MG, and U87 MG human glioblastoma cells. Values were normalized to Beta-actin, and statistics were calculated in GraphPad Prism. Cells were treated with TMZ 1 mM. CBD 20 μM, WFA 150 nM, or indicated combinations for 48 h prior to analysis by quantitative real-time PCR. ANOVA (Dunnett) n=3*=P≤0.05, **=P≤0.01, ***=P≤0.001.

FIG. 5 provides a scheme demonstrating cell viability assays of glioblastoma cells treated with varying concentrations of CBD and WFA. ATP activity of cells was quantified using luminescence and graphed. Calculations for the synergistic activity were done in CompuSyn and revealed synergy between certain concentrations of treatments. Dose-response of U87 and U251 cell lines to WFA and CBD single agents or combination after 48 hr treatment. Combination index for each dose was calculated using CompuSyn Software.

FIG. 6 provides the dose-response of U251 cell line to TMZ and CBD single agents or combination after 48 hr treatment. Combination index for each dose was calculated using CompuSyn Software.

FIG. 7 is a schematic depicting the downstream effects of CBD and WFA treatment on cells.

FIG. 8 is an outline of experimental design to investigate the effects of drug combinations.

FIG. 9 is a schematic depicting the differential outcomes of TMZ treatment depending on the activity of MGMT mediated repair.

FIGS. 10A-10E provide schemes demonstrating that CCR6/CCL20 knockdown improves CBD+TMZ therapy in vivo. C57BL/6 mice (n=6) were inoculated subcutaneously in the flank with 5×10⁵ CT2A cells on day 0. Treatments were started on day 16. Vehicle, 25 mg/kg CBD, 20 mg/kg TMZ, or combination with either ShScrambled DPX or ShCombo CCL20/CCR6 DPX and continued every 3rd day. Tumor size was monitored by vernier caliper. *=compared to control, #=compared to TMZ+CBD ANOVA with Holm Sidack p<0.05. At D33 tumors were excised and weighed. (FIG. 10A) Tumor growth measured on indicated days by caliper. ANOVA (Dunnett) *=P≤0.05. (FIG. 10B). Tumor weights measured on day 30. (FIGS. 10C-10D) At endpoint (D33) spleens were collected from drug or vehicle treated tumor bearing mice and splenocytes were subjected to immunophenotyping of myeloid cells by flow cytometry. n=5*=compared to control, #=compared to TMZ, ANOVA with Holm Sidack p<0.05. (FIG. 10E) Representative images of IHC of tumor sections. Histograms represent the quantification of expression (Intden/unit area). Data expressed as mean±SEM, Student's t-test, n=2/group. *=P0.05.

FIGS. 11A-11D provide schemes demonstrating the lipidomics of control and treated CT-2A flank tumors. (FIG. 11A) Procedural flowchart of total lipid extraction for MS/MS analyses. (FIG. 11B) Heatmap with hierarchical clustering displaying the relative abundance of lipid mediators or metabolites across different treatment groups. (FIG. 11C) PLS-DA biplot showing sample clustering along the first two components with loading vectors representing key lipid mediators and confidence ellipses indicating different treatment groups. (FIG. 11D) Stacked bar plots representing the relative abundance of HETEs, HEPE, EETs, and HDHA in different treatment groups and control tumors. Data representative of average n=5 mice per group.

FIGS. 12A-12B provide schemes demonstrating the viability assays of Spectrum Cannabidiol Oil via Pioneered nano-Emulsion (SCOPE) formulation. Cells were treated with TMZ and SCOPE nanoemulsion containing different doses of CBD in two different GBM cell lines, T-98G (FIG. 12A) and CT-2A (FIG. 12B). Tables show synergy or additive effects of SCOPE when combined with TMZ.

FIGS. 13A-13C provide schemes demonstrating that treatment with SCOPE significantly reduces Ki67 and IBA1 expression in CT-2A brain tumoroids. CT-2A glioblastoma-derived tumoroids were treated with control (no CBD) or SCOPE (5 μM CBD) for 48 hours. The tumoroids were cryo-sectioned and stained for Ki67 and IBA1 expression. (FIG. 13A) Immunofluorescent images representing Ki67 (upper panel) and IBA1 (lower panel) staining. Histogram depicting Ki67 (FIG. 13B) and IBA1 (FIG. 13C) expression (Intden/Unit area). Data are presented as mean±SEM (n=5/group). *=P≤0.05.

FIGS. 14A-14B provide images demonstrating the comparison of brain accumulation DiR nanoemulsions administered by intranasal (IN) or oral route at 3 h and 6 h post-delivery. The in vivo imaging system (IVIS) results of mice and excised brains showed that the IN route is substantially more effective than the oral route in delivering drugs to the brain. Intranasal delivery of nanoemulsion is more effective than oral administration. Mice (FIG. 14A) or brains (FIG. 14B) were imaged by IVIS 3 h or 6 h after administration of DiR nanoemulsion, either via IN or Oral route. n=2.

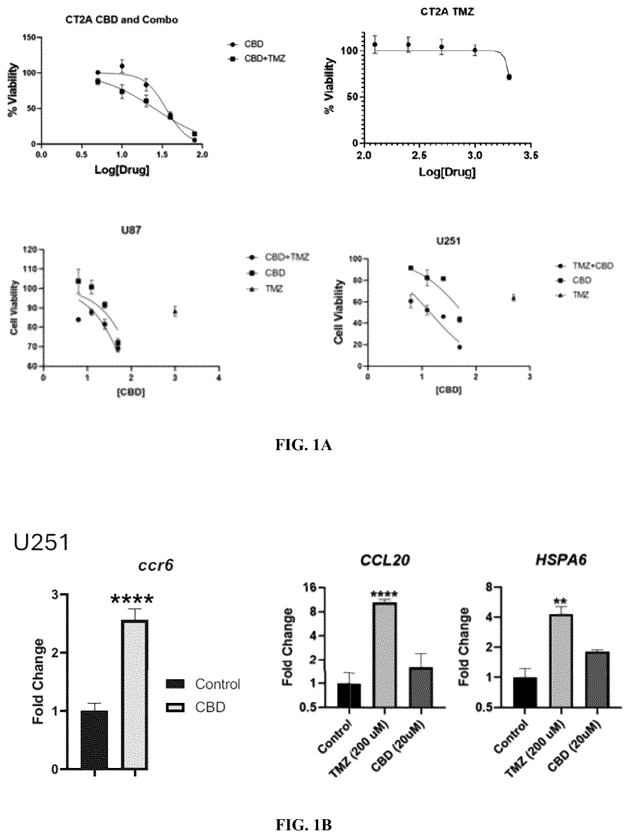

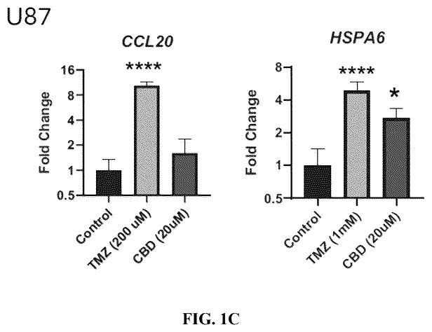

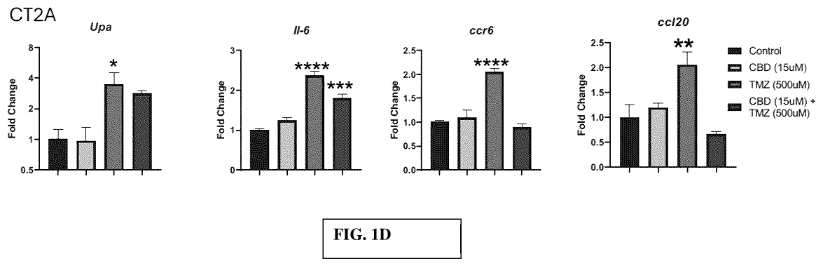

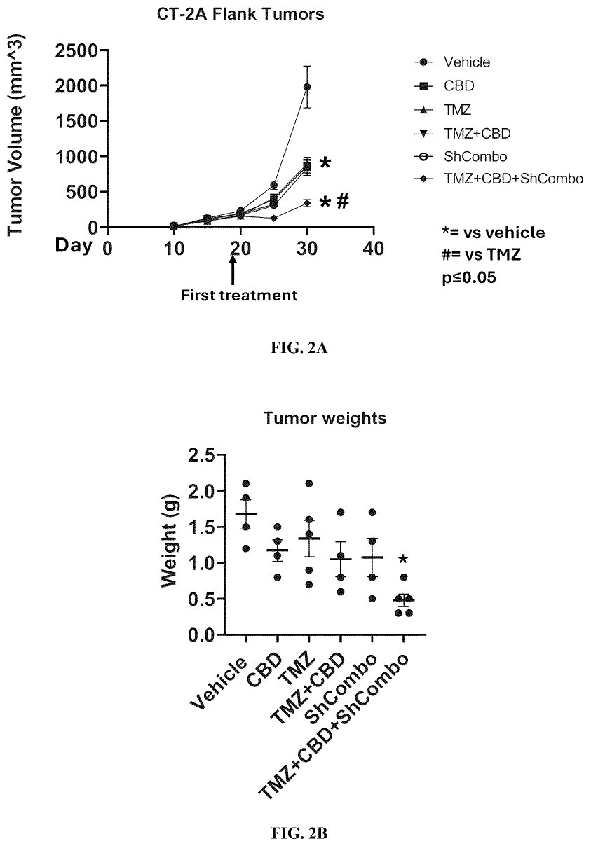

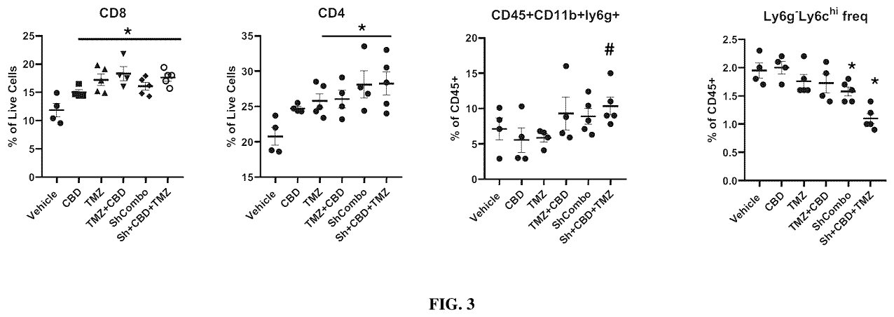

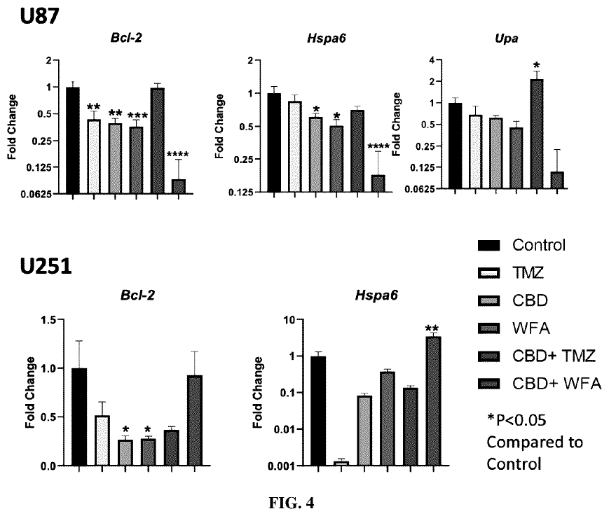

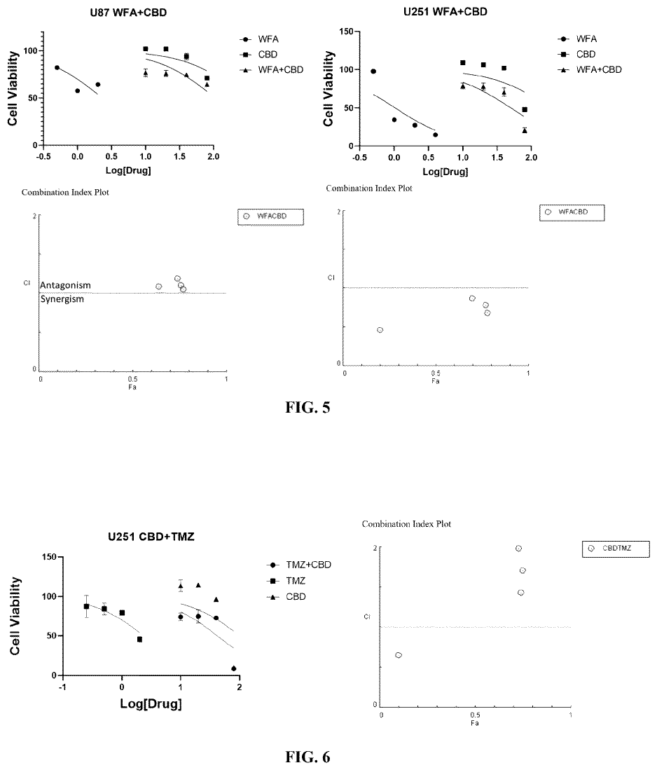

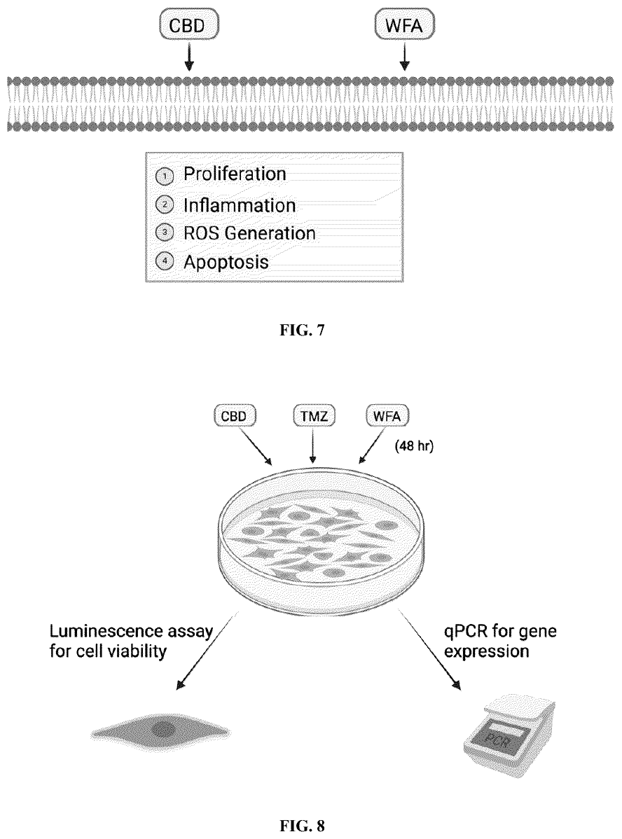

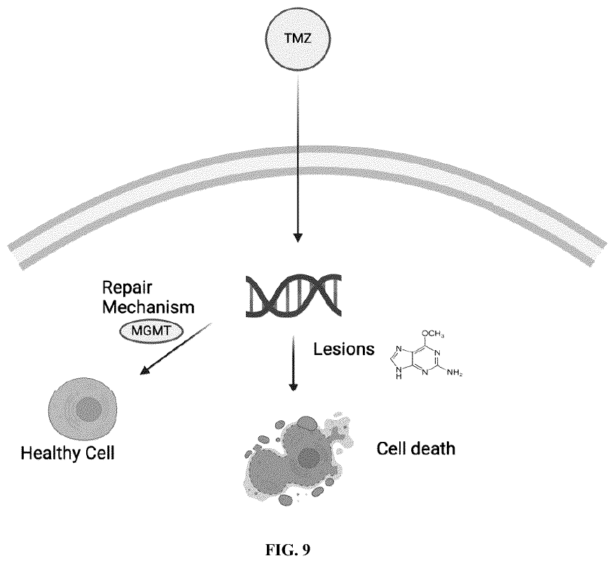

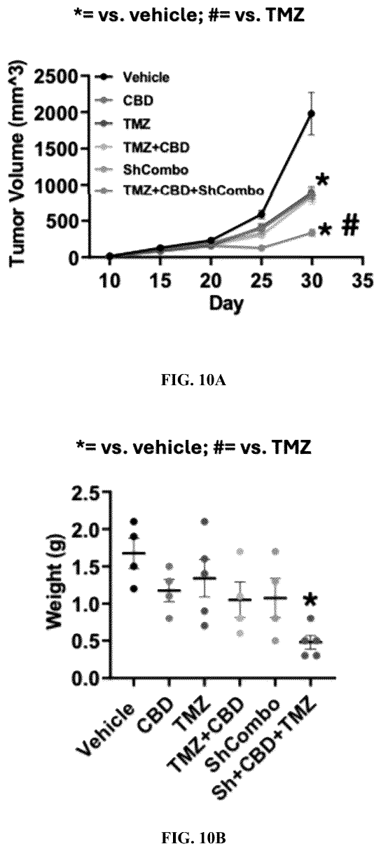

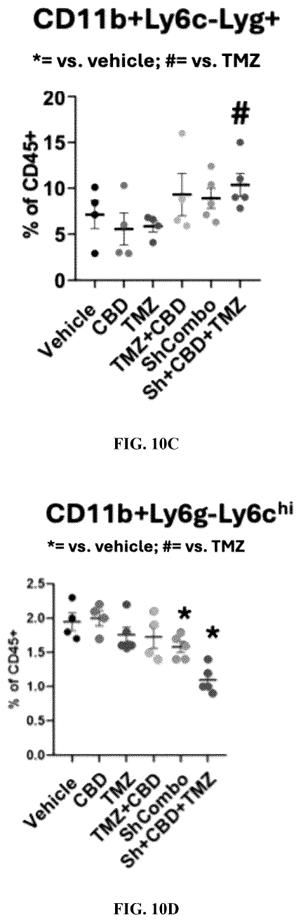

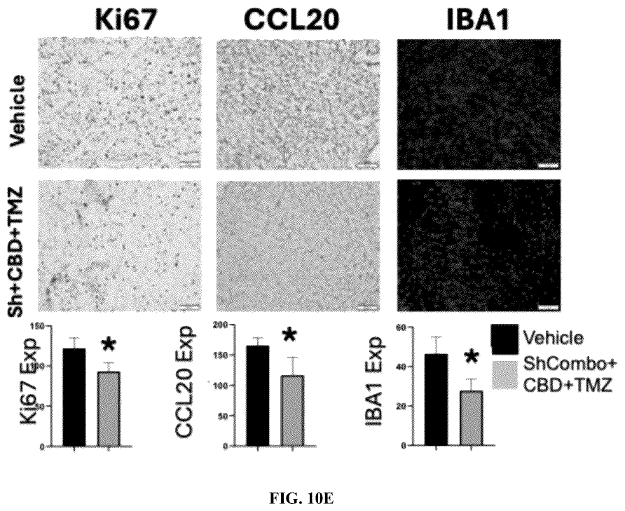

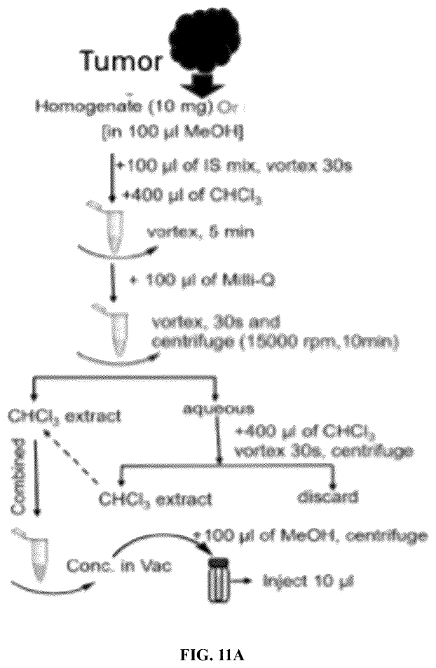

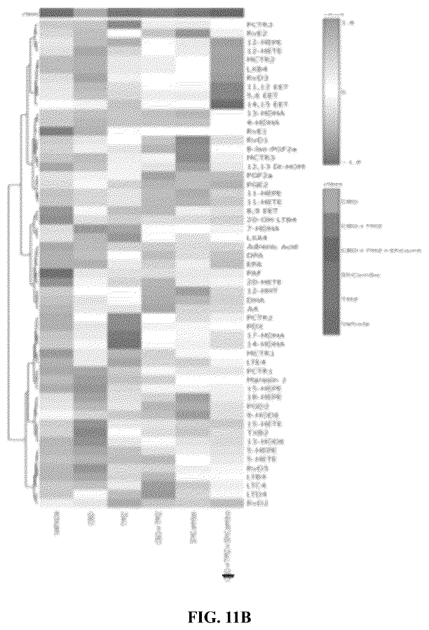

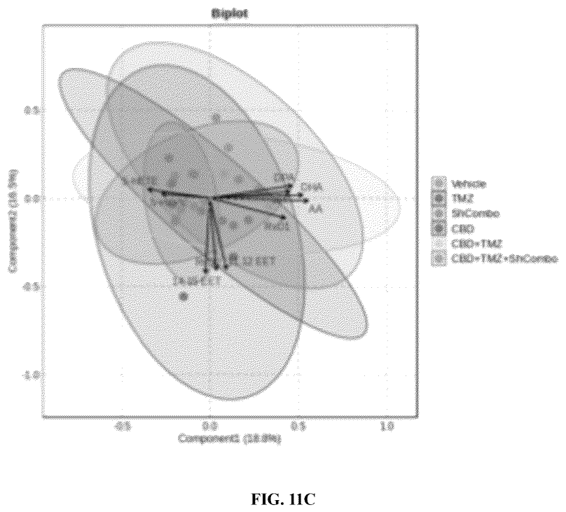

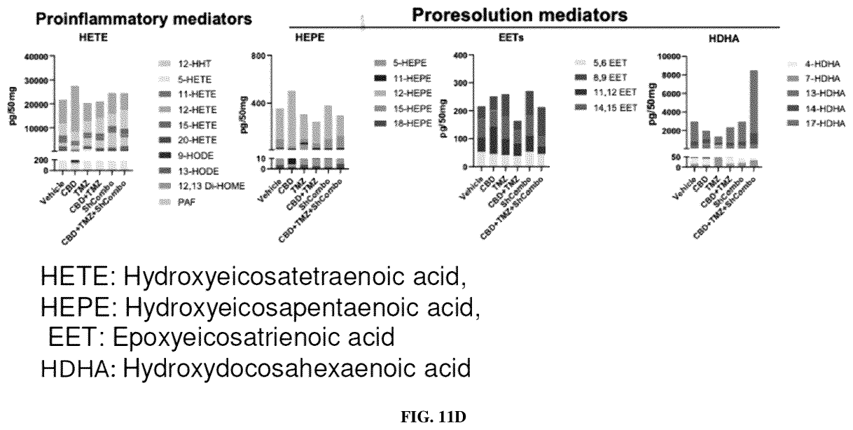

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular examples only and is not intended to be limiting.

### Definitions

As used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

It should be noted that amounts and other numerical data can be expressed herein in a range format. It can be further understood that the endpoints of each of the ranges are significant both in relation to the other endpoint and independent of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y’, and ‘about z’ as well as the ranges of ‘less than x,’ less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y′, and ‘greater than z.’ In addition, the phrase “about ‘x’ to ‘y’,” where ‘x’ and ‘y’ are numerical values, includes “about ‘x’ to about ‘y’.”

It is to be understood that such a range format is used for convenience and brevity and, thus, should be interpreted in a flexible manner to include not only the numerical values explicitly recited as the limits of the range but also all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

“Comprising” is intended to mean that the compositions, methods, etc. include the recited elements, but do not exclude others. “Consisting essentially of”′ when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate-buffered saline, preservatives, and the like. “Consisting of” shall mean excluding more than trace elements of other ingredients and substantial method steps for administering the compositions provided and/or claimed in this disclosure. Examples defined by each of these transition terms are within the scope of this disclosure.

A “control” is an alternative subject or sample used in an experiment for comparison purposes. A control can be “positive” or “negative.”

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, or composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

An “increase” can refer to any change that results in a greater amount of a symptom, disease, composition, condition, or activity. An increase can be any individual, median, or average increase in a condition, symptom, activity, or composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Optional” or “optionally” means that the subsequently described event or circumstance may or may not occur, and that the description includes instances where said event or circumstance occurs and instances where it does not.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing a physical phenomenon. For example, an event or change in environment is sensed, and a signal output is released in the form of light.

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

As used herein, “IC₅₀” is intended to refer to the concentration or dose of a substance (e.g., a compound or a drug) that is required for 50% inhibition or diminuation of a biological process, or component of a process, including a protein (e.g., CCR6 or CCL20), subunit, organelle, ribonucleoprotein, etc. IC₅₀ also refers to the concentration or dose of a substance that is required for 50% inhibition or diminuation in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest, such as obesity or diabetes. As appropriate, the response can be measured in a transgenic or knockout mouse or rat wherein a gene or genes have been introduced or knocked-out, as appropriate, to replicate a disease process.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

An “adjuvant” refers to a drug, molecule, substance, or a combination thereof that is used to increase the efficacy or potency of certain therapeutic agents, such as for example vaccines and/or antibodies. “Adjuvant(s)” are often at least one ingredient used in some vaccines that help create a stronger immune response in the host receiving said vaccine.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Therapeutic agent” refers to any composition that has a beneficial biological effect. Therapeutic agent includes, but is not limited to, genes and gene products, peptides and proteins, nanoparticles, nanoemulsions, lipids, carbohydrates, and small molecule drugs. In some examples, a therapeutic agent can be shRNAs encoding CCL20 and/or CCR6 delivered to the cell in a DNA plasmid complexed to a dendrimer to form a dendriplex. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising a therapeutic agent) refers to an amount that is effective to achieve a desired therapeutic result. In some examples, a desired therapeutic result is the control of CBD and/or TMZ refractory glioblastoma. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “anticancer” and “anticarcinogen” refer to a substance, composition, or formula that counteracts the effects or inhibits the development of a cancerous cells and tissues.

The term “cancer” is used to address any neoplastic disease and is not limited to epithelial neoplasms (surface and glandular cancers; such a squamous cancers or adenomas)). It is used here to describe both solid tumors and hematologic malignancies, including epithelial (surface and glandular) cancers, soft tissue and bone sarcomas, angiomas, mesothelioma, melanoma, lymphomas, leukemias and myeloma.

The terms “immunotherapy” and “immunotherapeutic” refer to the treatment of disease by activating or suppressing the immune system. In cancer treatment, the most effective immunotherapies are cell-based immunotherapies that utilize lymphocytes, macrophages, dendritic cells, natural killer cells, cytotoxic T lymphocytes, etc. to defend the body against cancer by targeting abnormal antigens expressed on the surface of tumor cells.

Targeted therapy is a type of cancer treatment that uses a drug, substance, composition or formula that precisely identifies and targets proteins that control how cancer cells grow, divide, and spread and then attacks/kills the cancer cell. A targeted therapy can be used by itself or in combination with other anti-cancer treatments, such as, for example chemotherapy, surgery, or radiation therapy.

“Expression” as used herein refers to the process by which information from a gene is used in the synthesis of a functional gene product that enables it to produce a peptide/protein end product, and ultimately affect a phenotype, as the final effect.

The word “vector” refers to any vehicle that carries a polynucleotide into a cell for the expression of the polynucleotide in the cell. The vector can be, for example, a plasmid, a virus, a phage particle, or a nanoparticle. Once transformed into a suitable host, the vector can replicate and function independently of the host genome, or can in some instances, integrate into the genome itself. In some examples, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In some examples, the expression vector comprises a plasmid. A plasmid can be capable of extrachromosomal replication or, optionally, can integrate into the host genome. In some examples, the compositions disclosed herein can include a plasmid encoding shRNA that targets CCL20 and/or CCR6. As used herein, the term “integrated” used in reference to an expression vector (e.g., a plasmid or viral vector) means the expression vector, or a portion thereof, is incorporated (physically inserted or ligated) into the chromosomal DNA of a host cell.

The term “nucleic acid” as used herein can be double-stranded, single-stranded, or contain portions of both double and single stranded sequence. If the nucleic acid is single-stranded, the sequence of the other strand is also identifiable and thus the definition includes the complement of the sequence disclosed.

“Short hairpin RNA” or “shRNA” as used herein refers to single stranded RNA molecules that are constructed by connecting sense and antisense strands of an siRNA duplex with a loop sequence, thus allowing a single transcript to fold back on a duplex structure upon being transcribed. After transcription, the shRNA molecules are processed into siRNAs by the Dicer enzyme and are capable of suppressing a gene. In some examples, the compositions disclosed herein can knock down, or silence, the genes CCL20, CCR6, or a combination thereof.

As used herein, the term “cannabis” encompasses all types of cannabis, including wild type Cannabis sativa, Cannabis chemovars, Cannabis indica, Cannabis ruderalis, and variants, hybrids, or cultivars thereof. As used herein, the term “cannabinoid” refers to a terpene (e.g., an organic compound built from isoprene subunits) which can interact with cannabinoid receptor 1 (CB₁) or cannabinoid receptor 2 (CB₂) in a human.

Cannabinoids include “phytocannabinoids,” which are produced by the cannabis plant, “endocannabinoids,” which are produced by mammals, and synthetic cannabinoids, however, the term “cannabinoid” is primarily used herein to refer to phytocannabinoids. Examples of cannabinoids include cannabinol (CBN), cannabinolic acid (CBNA), tetrahydrocannabinol (THC), tetrahydrocannabinolic acid (THCA), cannabidiol (CBD), cannabinolic acid (CBDA), tetrahydrocannabivarin (THCV), tetrahydrocannabinol acetate ester (THCOA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabidivarin (CBDV), cannabichromevarin (CBCV), cannabigerovarin (CBGV), cannabigerol monomethyl ether (CBGM), β-caryophyllene, β-caryophyllene oxide, or any combination thereof. It is understood that reference to cannabinoids includes reference to all relevant isomers. For example, reference to CBD includes reference to all relevant isomers of CBD, including, but not limited to delta-8-CBD, delta-9-CBD, delta-10-CBD, and delta-11-CBD. Cannabinoids as disclosed herein can also include full-spectrum or broad-spectrum formulations. “Full spectrum” includes all phytochemicals found in cannabis, including cannabinoids, non-cannabinoid terpenes, and flavonoids. Similarly, “Broad spectrum” includes all non-cannabinoid terpenes, flavinoids, and cannabinoids found in cannabis except for THC, which can be absent or present in non-detectable amounts. In some examples, the cannabinoid can be broad-spectrum.

### Compositions/Compounds

Disclosed herein are compositions for treating CBD- and/or TMZ-refractory glioblastoma, comprising a cannabinoid, temozolomide (TMZ), at least one CCL20/CCR6 inhibitor, or any combination thereof.

### Cannabis and Cannabinoids

Phytochemicals such as Cannabidiol (CBD) and/or Withaferin A (WFA), a highly cytotoxic constituent of the Ayuvedic herb Ashwagandha (Withania somnifera), are potential therapeutics for glioblastoma due to their anti-cancer properties, such as inducing apoptosis, inhibiting cell proliferation, and suppressing tumor invasion in pre-clinical studies of many cancers. Synergy was shown between WFA and CBD, showing potential as a therapeutic to improve treatment efficacy. Cannabinoids can serve as potential therapeutics for glioblastoma due to their reported anti-cancer properties, such as inducing apoptosis, inhibiting cell proliferation, and suppressing tumor invasion in pre-clinical studies of many cancers. In some examples, the compositions disclosed herein can include a cannabinoid. In some examples, the cannabinoid can be CBN, CBNA, THC, THCA, CBD, CBDA, THCV, THCOA, CBG, CBGA, CBC, CBCA, CBL, CBLA, CBV, CBDV, CBCV, CBGV, CBGM, β-caryophyllene, β-caryophyllene oxide, pharmaceutically acceptable salts thereof, or any combination thereof. In some examples, the cannabinoid can be CBD or a pharmaceutically acceptable salt thereof.

### Temozolomide

Temozolomide (TMZ) is a chemotherapeutic agent that induces cell cycle arrest and is the gold standard therapeutic for treating glioblastoma. While TMZ has limited efficacy in GBM due to drug resistance and poor clinical response rates, a combination of TMZ with other therapeutic agents, such as cannabinoids and/or WFA, can lead to a more synergistic effect, such as increased oxidative stress on cells. Furthermore, a combination of CBD and/or WFA with treatments for GBM, such as temozolomide (TMZ), can yield a synergistic effect. In some examples, the compositions disclosed herein can include temozolomide or related alkylating agents (e.g., lomustine, carmustine).

TMZ can be given in an amount that is indicated for a particular patient, taking into consideration factors such as indication, body size, blood count, liver function, whether radiation is also to be used, and the like, which can be determined by one of skill in the art. In specific example, TMZ is given at a dose less than that which would normally be indicated for a particular patient. For example, TMZ can be given at from 75 to 200 mg/m², or TMZ can be give at from 25 to 100 mg/m², from 25 to 75 mg/m², from 50 to 150 mg/m², from 50 to 100 mg/m², or from 75 to 100 mg/m².

### CCL20/CCR6 Inhibitors

Chemokine (C-C motif) ligand 20 (CCL20) is a protein that is involved in immunoregulatory and inflammatory processes. CCL20 acts by binding and activating the chemokine receptor 6 (CCR6) present on the surface of the inflammatory cells. CCR6 activation has been linked to tumor survival signaling, progression, metastasis, and poor clinical outcomes in glioblastoma. Furthermore, CBD mono- or combination therapy disclosed herein can increase the expression/activation of the CC chemokine ligand 20 (CCL20) and its receptor (CCR6) axis in glioblastoma tumor models. In some examples, the compositions disclosed herein include a CCL20/CCR6 inhibitor.

In some examples, the CCL20/CCR6 inhibitor can be a small molecule inhibitor, such as CCR6 inhibitor 1, CCR6 antagonist 1, PF-07054894, IDOR-1117-2520, (S)-IDOR-1117-2520, CCR6 antagonist 4 (OXM1), CCR6 antagonist 3 (OXM2), SQA1, Takeda Cpd 35, Takeda Cpd 1, OXM1, OXM2, Roche CCR6 antagonist, ChemoCentryx CCR6 antagonist 1, ChemoCentryx CCR6 antagonist 2, pharmaceutically acceptable salts thereof, or any combination thereof.

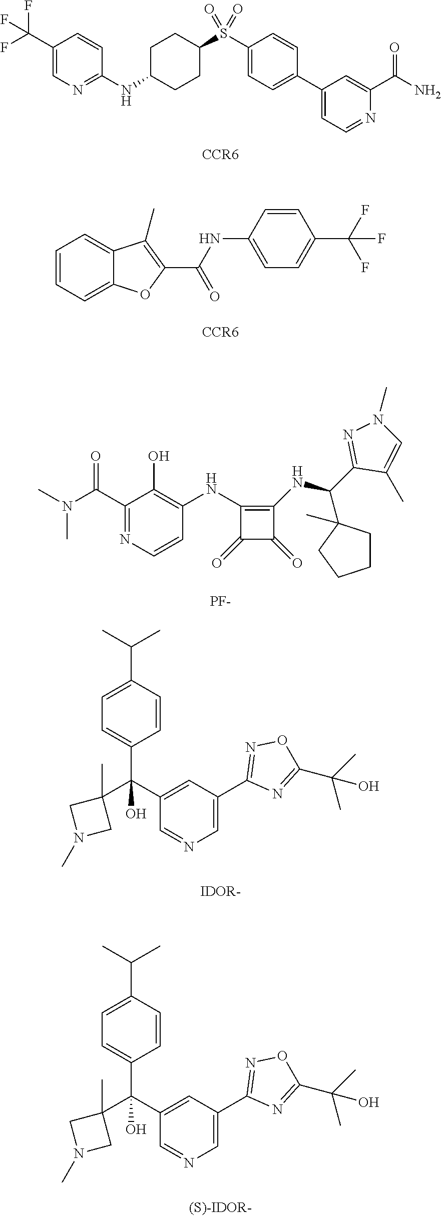

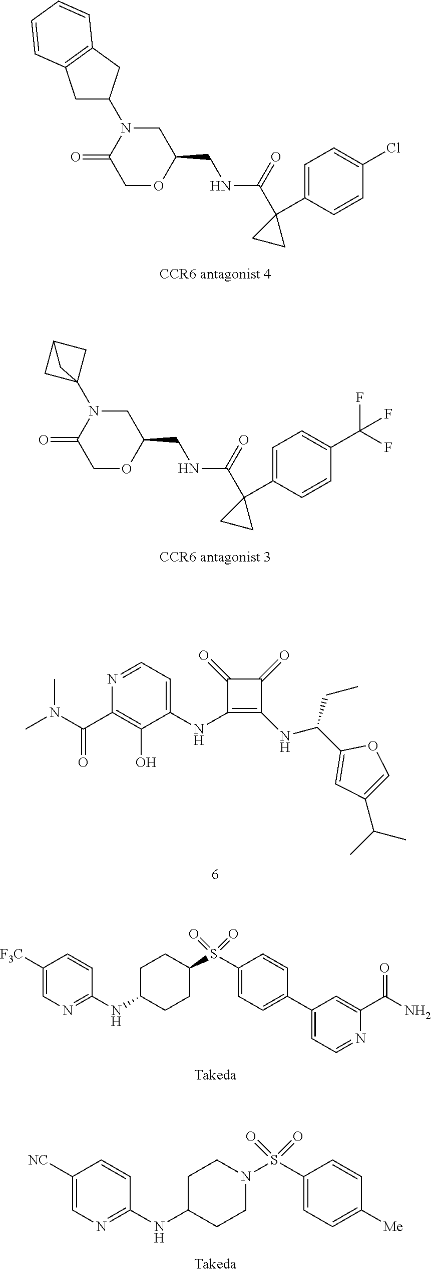 [SMILES: O=C1CO[C@H](CNC(=O)C2(c3ccc(Cl)cc3)CC2)CN1C1Cc2ccccc2C1 ; O=C1CO[C@H](CNC(=O)C2(c3ccc(C(F)(F)F)cc3)CC2)CN1C12CC(C1)C2 ; CC[C@@H](Nc1c(Nc2ccnc(C(=O)N(C)C)c2O)c(=O)c1=O)c1cc(C(C)C)co1 ; "6" ; NC(=O)c1cc(-c2ccc(S(=O)(=O)[C@H]3CC[C@H](Nc4ccc(C(F)(F)F)cn4)CC3)cc2)ccn1 ; Cc1ccc(S(=O)(=O)N2CCC(Nc3ccc(C#N)cn3)CC2)cc1]

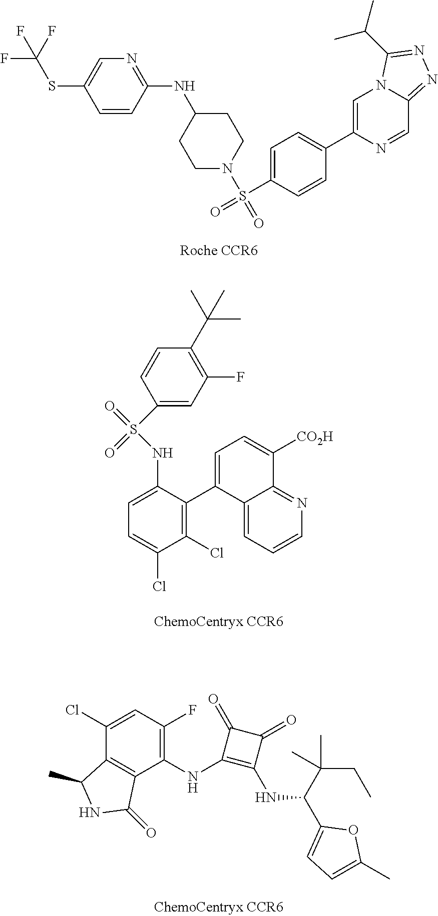

In some examples, the CCL20/CCR6 inhibitor can be a nucleic acid. In some examples, the nucleic acid is an antisense compound. In some examples, the antisense compound is selected from the group consisting of an antisense oligonucleotide, a small interfering RNA (siRNA), microRNA (miRNA), short hairpin (shRNA), a ribozyme, an immune-stimulating nucleic acid, an antagomir, an antimir, a microRNA mimic, a supermir, a U1 adaptor, and an aptamer. In some examples, the nucleic acid can be expressed by a vector such as a plasmid. In some examples, the vector can be conjugated to a nanoparticle. In some examples, the nanoparticle can be a dendrimer. “Dendrimer” as used herein refers to highly branched synthetic polymer macromolecules capable of being used in the delivery of therapeutic agents to a patient. Dendrimers as used herein refer to nanoparticle dendrimers having a size range of 5-50 nm (e.g., from about 5-25 nm, 25-50 nm, 5-15 nm, 15-25 nm, 25-35 nm, 35-50 nm, 5-10 nm, 10-20 nm, 20-30 nm, 30-40 nm, 40-50 nm, 10-15 nm, 15-20 nm, 20-25 nm, 25-30 nm, 30-35 nm, 35-40 nm, 40-45 nm, or 45-50 nm). In some examples, dendrimers can be about 5 nm. Dendrimers are constructed by the successive addition of branching group layers, with each branching group layer being a new generation. Dendrimers capable of being used herein include, but are not limited to, poly(propyleneimine) dendrimers (PPI), polyether (PE) dendrimers, phenyl acetylene dendrimers, glycodendrimers, carbohydrate dendrimers, metallodendrimers, fulleropyrrolidine dendrimers, carbosilane dendrimers, poly(amidoamine) dendrimers (PAMAM), poly 2,2-bis(methylol) propionic acid (PBisMPA), poly(benzyl ether) dendrimers (PBzE), poly(lysine) dendrimers (PLL), or polymelamine (triazine) dendrimers.

“Dendriplex” as used herein refers to dendrimers that have been functionalized by the complexation (conjugation) of at least one therapeutic agents and/or genes to deliver genes/gene products and/or therapeutic agents to the brain and/or to glioblastoma tumors. In some examples, the dendrimers have at least one shRNA-encoding DNA vector complexed to the outer surface of the dendrimer. In some examples, the shRNA contained within the vector encodes chemokine ligand 20 (CCL20), chemokine receptor 6 (CCR6), or a combination thereof. In some examples, the dendriplexes can have a size of about 50-200 nm (e.g., from about 50-150 nm, 150-200 nm, 50-100 nm, 100-150 nm, 150-200 nm, 50-75 nm, 75-100 nm, 100-125 nm, 125-150 nm, 150-175 nm, or 175-200 nm). In some examples, the dendriplexes can have a size of about 100 nm.

PAMAM dendrimers have been widely used in the last decade for diagnostic and therapeutic applications due to their ability to efficiently cross the BBB to deliver drugs to the brain parenchyma. Their unique hyper-branched structure allows for multiple modifications and functionalities, as well as for high drug-loading capacity. The amine end of PAMAM polymers is a useful gene therapy tool for brain diseases as it can be easily complexed with DNA or RNA via electrostatic interactions. If PAMAM dendrimers are used, any of generations 0-11 (GO-G11) can be used. In some examples, the compositions disclosed herein can include PAMAM G4. PAMAM dendrimers generally comprise an ethylenediamine core, a repetitive branching amidoamine internal structure and a primary amine terminal surface. In some examples, the dendrimer nanoparticle comprises PAMAM.

### Cannabinoid+PUFAs-Nanoemulsions

The compositions and methods disclosed herein also pertain to the identification of lipids that provide protection from glioblastoma and neuroinflammation. In some examples, the cannabinoids disclosed herein can form a nano-emulsion with a polyunsaturated fatty acid. Polyunsaturated fatty acids (PUFAs) are fatty acids characterized by a backbone containing two or more alkenes and can mediate cell proliferation and apoptosis in cancer cells. In some examples, the compositions disclosed herein can include PUFAs, such as omega-3 fatty acids (e.g., docosahexaenoic acids (DHAs), hydroxydocosahexaenoic acids (HDHAs), alpha-linoleic acid (ALAs), eicosapentaenoic acids (EPAs), hydroxyeicosapentaenoic acids (HEPEs), stearidonic acids (SDAs), eicosatetraenoic acids (ETAs), hydroxyeicosatetraenoic acids (HETEs), docosapentaenoic acids (DPA), tetracosahexaenoic acids, or derivatives thereof), omega-6 fatty acids (e.g., arachidonic acids (ARAs), epoxyeicosatrienoic acids (EETs), linoleic acids, gamma-linoleic acids (GLAs), calendic acids, eicosadienoic acids, dihomo-gamma-linoleic acids (DGLAs), docosadienoic acids, adrenic acids, osbond acid, tetracosatetraenoic acids, tetracosapentaenoic acids, or derivatives thereof), or any combination thereof.

Furthermore, a lipidomics analysis indicates that the accumulation of HDHA family of lipids plays a critical role in the attenuation of glioblastoma. To harness the synergy between TMZ and CBD, a new formulation of spectrum CBD with HDHA rich fish oil as high-impact nanoscale (e.g., 30-100 nm) emulsion was developed, referred to as SCOPE. It was found that SCOPE treatment reduced the viability of GBM cells and CT-2A organoids growth. In some examples, the cannabinoid disclosed herein can form a nano-emulsion with a HDHA, such as 4-HDHA, 7-HDHA, 13-HDHA, 14-HDHA, or any combination thereof. In some examples, the HDHA can be 17-HDHA, 14-HDHA, or a combination thereof.

### Methods

It is understood and herein contemplated that the disclosed compositions comprising a cannabinoid, TMZ, a CCL20/CCR6 inhibitor, or any combination thereof or pharmaceutically acceptable salts thereof can be used to treat CBD- and/or TMZ-refractory glioblastoma. Thus, in one example disclosed herein are methods of treating, inhibiting, reducing, decreasing, ameliorating, and/or preventing a CBD- and/or TMZ-resistant glioblastoma or metastasis in a subject comprising administering to the subject a therapeutically effective amount of the compositions of a cannabinoid, TMZ, a CCL20/CCR6 inhibitor, or any combination thereof or pharmaceutically acceptable salts thereof disclosed herein. For example, disclosed herein are methods of treating, inhibiting, reducing, decreasing, ameliorating, and/or preventing a CBD- and/or TMZ-resistant glioblastoma and/or metastasis in a subject comprising administering to the subject a therapeutically effective amount of a composition comprising a cannabinoid (such as, for example CBD), TMZ, a CCL20/CCR6 inhibitor (such as, for example, a CCL20/CCR6 dendriplex), or any combination thereof or pharmaceutically acceptable salts thereof.

The pathophysiology of GBM includes uncontrolled cell proliferation, angiogenesis (or rapid blood vessel formation and spreading), necrosis, hypoxia, and high tumor infiltration, thereby spreading easily. Furthermore, GBM is believed to arise from mutations occurring within supportive cells of the brain known as astrocytes. In some examples, a therapeutically effective amount can be any amount that decreases tumor survival signaling, inhibits metastasis progression, inhibits uncontrolled tumor cell proliferation, inhibits angiogenesis, induces tumor apoptosis, suppresses tumor invasion, induces tumor cell cycle arrest, increases T cell proliferation, or reduces the expression of glioblastoma tumor cell markers or gene targets (e.g., MGMT, Bcl-2, Hspa6, Upa, Ki67 or IBA1).

In some examples, the therapeutically effective amount of CBD or a pharmaceutically acceptable salt thereof in the compositions and methods disclosed herein can be from about 5 mg/kg to 50 mg/kg. In some examples, the therapeutically effective amount of CBD or a pharmaceutically acceptable salt thereof in the compositions and methods disclosed herein can be about 25 mg/kg.

In some examples, the therapeutically effective amount of TMZ or a pharmaceutically acceptable salt thereof in the compositions and methods disclosed herein can be from about 5 mg/kg to 50 mg/kg. In some examples, the therapeutically effective amount of CBD or a pharmaceutically acceptable salt thereof in the compositions and methods disclosed herein can be about 20 mg/kg.

In some examples, the therapeutically effective amount of a CCR6/CCL20 inhibitor or a pharmaceutically acceptable salt thereof in the compositions and methods disclosed herein can be a concentration or dose that is required for 50% inhibition or diminuation of CCR6 or CCL20.

In some examples, the therapeutically effective amount can be from about 5 nM-3500 nM (e.g., from about 5 nM-3000 nM, 3000-3500 nM, 5 nM-1500 nM, 1500 nM-3000 nM, 5 nM-1000 nM, 1000 nM-2000 nM, 2000-3000 nM, 5 nM-500 nM, 500 nM-1000 nM, 1000 nM-1500 nM, 1500 nM-2000 nM, 2000 nM-2500 nM, 2500 nM-3000 nM, 5 nM-250 nM, 250 nM-500 nM, 500 nM-750 nM, 750 nM-1000 nM, 1000 nM-1250 nM, 1250 nM-1500 nM, 1500 nM-1750 nM, 1750 nM-2000 nM, 2000 nM-2250 nM, 2250 nM-2500 nM, 2500 nM-2750 nM, 2750 nM-3000 nM, 3000 nM-3250 nM, 3250 nM-3500 nM, 5 nM-150 nM, 150 nM-300 nM, 300 nM-450 nM, 450 nM-600 nM, 600 nM-750 nM, 750 nM-900 nM, 900 nM-1050 nM, 1050 nM-1200 nM, 1200 nM-1350 nM, 1350 nM-1500 nM, 1500 nM-1650 nM, 1650 nM-1800 nM, 1800 nM-1950 nM, 1950 nM-2100 nM, 2100 nM-2250 nM, 2250 nM-2400 nM, 2400 nM-2550 nM, 2550 nM-2700 nM, 2700 nM-2850 nM, 2850 nM-3000 nM, 3000 nM-3150 nM, 3150 nM-3300 nM, 3300 nM-3500 nM, 5-100 nM, 100-200 nM, 200-300 nM, 300-400 nM, 400-500 nM, 500-600 nM, 600-700 nM, 700-800 nM, 800-900 nM, 900-1000 nM, 1000-1100 nM, 1100-1200 nM, 1200-1300 nM, 1300-1400 nM, 1400-1500 nM, 1500-1600 nM, 1600-1700 nM, 1700-1800 nM, 1800-1900 nM, 1900-2000 nM, 2000-2100 nM, 2100-2200 nM, 2200-2300 nM, 2300-2400 nM, 2400-2500 nM, 2500-2600 nM, 2600-2700 nM, 2700-2800 nM, 2800-2900 nM, 2900-3000 nM, 3000-3100 nM, 3100-3200 nM, 3200-3300 nM, 3300-3400 nM, 3400-3500 nM, 5-50 nM, 50-100 nM, 100-150 nM, 150-200 nM, 200-250 nM, 250-300 nM, 300-350 nM, 350-400 nM, 400-450 nM, 450-500 nM, 500-550 nM, 550-600 nM, 600-650 nM, 650-700 nM, 700-750 nM, 750-800 nM, 800-850 nM, 850-900 nM, 900-950 nM, 950-1000 nM, 1000-1050 nM, 1050-1100 nM, 1100-1150 nM, 1150-1200 nM, 1200-1250 nM, 1250-1300 nM, 1300-1350 nM, 1350-1400 nM, 1400-1450 nM, 1450-1500 nM, 1500-1550 nM, 1550-1600 nM, 1600-1650 nM, 1650-1700 nM, 1700-1750 nM, 1750-1800 nM, 1800-1850 nM, 1850-1900 nM, 1900-1950 nM, 1950-2000 nM, 2000-2050 nM, 2050-2100 nM, 2100-2150 nM, 2150-2200 nM, 2200-2250 nM, 2250-2300 nM, 2300-2350 nM, 2350-2400 nM, 2400-2450 nM, 2450-2500 nM, 2500-2550 nM, 2550-2600 nM, 2600-2650 nM, 2650-2700 nM, 2700-2750 nM, 2750-2800 nM, 2800-2850 nM, 2850-2900 nM, 2900-2950 nM, 2950-3000 nM, 3000-3050 nM, 3050-3100 nM, 3100-3150 nM, 3150-3200 nM, 3200-3250 nM, 3250-3300 nM, 3300-3350 nM, 3350-3400 nM, 3400-3450 nM, or 3450-3500 nM).

In some examples, the therapeutically effective amount can be about 6 nM. In some examples, the therapeutically effective amount can be about 5.7 nM. In some examples, the therapeutically effective amount can be about 30 nM. In some examples, the therapeutically effective amount can be about 63 nM. In some examples, the therapeutically effective amount can be about 158 nM. In some examples, the therapeutically effective amount can be about 166 nM. In some examples, the therapeutically effective amount can be about 250 nM. In some examples, the therapeutically effective amount can be about 3100 nM.

In some examples, the therapeutically effective amount can be from about 0.5 mg/kg-20 mg/kg (e.g., from about 0.5 mg/kg-15 mg/kg, 15 mg/kg-20 mg/kg, 0.5 mg/kg-10 mg/kg, 10 mg/kg-20 mg/kg, 0.5 mg/kg-5 mg/kg, 5 mg/kg-20 mg/kg, 0.5 mg/kg-1 mg/kg, 1 mg/kg-20 mg/kg, 0.5 mg/kg-0.75 mg/kg, 0.75 mg/kg-20 mg/kg, 10 mg/kg-15 mg/kg, 5 mg/kg-10 mg/kg, 5 mg/kg-15 mg/kg, 1 mg/kg-5 mg/kg, 1 mg/kg-10 mg/kg, 1 mg/kg-15 mg/kg, 0.75 mg/kg-1 mg/kg, 0.75 mg/kg-5 mg/kg, 0.75 mg/kg-10 mg/kg, 0.75 mg/kg-15 mg/kg).

In some examples, the therapeutically effective amount can be about 1 mg/kg.

Because glioblastoma cell lines show increased sensitivity to combination treatment with TMZ and CBD nano-emulsion compared to TMZ or CBD nano-emulsion alone, a sub-standard dose of TMZ or CBD nano-emulsion can be considered therapeutically effective when used in combination. A “sub-standard” dose or concentration is an amount that is insufficient to achieve a particular therapeutic effect, such as tumor viability. For example, if the IC₅₀ of a CBD nano-emulsion in a tumor cell line is 84 μM, then a sub-standard concentration of the CBD nano-emulsion can be 58 μM when dosed with TMZ. In some examples, a sub-standard dose or concentration can be less than 58 μM of the CBD nano-emulsion.

The administration of the disclosed compositions can occur in any order, simultaneously, or they can be administered sequentially, wherein one is given before the other. In some examples, the cannabinoid, TMZ, the CCL20/CCR6 inhibitor, or any combination thereof or pharmaceutically acceptable salts thereof can be administered in a single pharmaceutically acceptable composition or formulation. In some examples, the cannabinoid, TMZ, the CCL20/CCR6 inhibitor, or any combination thereof or pharmaceutically acceptable salts thereof can be administered in separate pharmaceutically acceptable compositions or formulations. In some examples, the separate pharmaceutically acceptable compositions or formulations can be administered sequentially. In some examples, the separate pharmaceutically acceptable compositions or formulations can be administered simultaneously.

Further provided herein are methods of treating or preventing CBD- and/or TMZ-resistant glioblastoma in a subject, comprising administering to the subject a therapeutically effective amount of a composition as disclosed herein. The methods can further comprise administering a second compound or composition, such as, for example, anticancer agents or anti-inflammatory agents. The administration of the disclosed compositions alone or in combination with a second compound or composition can occur in any order, simultaneously, or they can be administered sequentially, wherein one is given before the other. Additionally, the method can further comprise administering an effective amount of ionizing radiation to the subject. In some examples, the method can include administering the composition as disclosed herein in combination with anti-cancer agents for targeted therapy. In some examples, the targeted therapy can be chemotherapy. In some examples, the targeted therapy can be radiation therapy. In some examples, the targeted therapy can be an immunotherapy. In some examples, the targeted therapy can be surgery.

It is understood and herein contemplated that the disclosed compositions can be used alone or in combination with any anti-cancer agents known in the art including, but not limited to 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR.

In some examples, the anti-cancer agent can be a chemotherapy agent. In some examples, the chemotherapy agent can be altretamine, bendamustine, busulfan, carboplatin, chlorambucil, cisplatin, cyclophosphamide, dacarbazine, ifosfamide, mechlorethamine, melphalan, oxaliplatin, procarbazine, temozolomide, thiotepa, trabectedin, carmustine, lomustine, streptozocin, 5-fluorouracil, 6-mercaptopurine, azacitidine, capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, etoposide, irinotecan, irinotecan liposomal, mitoxantrone, teniposide, topotecan, cabazitaxel, docetaxel, nab-paclitaxel, paclitaxel, vinblastine, vincristine, vincristine liposomal, vinorelbine, daunorubicin, doxorubicin, doxorubicin liposomal, epirubicin, idarubicin, mitoxantrone, valrubicin, bleomycin, dactinomycin, mitomycin-c, all-trans-retinoic acid, arsenic trioxide, asparaginase, eribulin, ixabepilone, mitotane, omacetaxine, pegaspargase, procarbazine, romidepsin, vorinostat, dexamethasone, hydrocortisone, methylprednisolone, prednisolone, prednisone, or any combination thereof.

Methods of killing a tumor cell are also provided herein. The methods comprise contacting a tumor cell with an effective amount of a composition as disclosed herein. The methods can further include administering a second compound or composition (e.g., an anti-cancer agent or an anti-inflammatory agent) or administering an effective amount of ionizing radiation to the subject. The administration of the disclosed compositions alone or in combination with a second compound or composition can occur in any order, simultaneously, or they can be administered sequentially, wherein one is given before the other.

While little is known of risk factors associated with GBM, risk factors can include correlations with age, radiation exposure, environmental factors, and genetic mutations characterized with P53, PTEN, EGFR, PDGFR, TERT, and more. The method can include administering the composition directly after removal of a primary tumor, upon detection of regions of tumor cells at the preangiogenic stage, upon diagnosis of high-risk factors in the subject, or upon detection of certain cancer proteins in serum.

In some examples, disclosed are methods for treating a CBD- and/or TMZ-refractory tumor or tumor metastases in a subject by the administration to the subject a therapeutically effective amount of the compositions disclosed herein and at least one cancer immunotherapeutic agent. The disclosed compositions can be administered alone or in combination with a cancer immunotherapeutic agent. The administration of the disclosed compositions alone or in combination with a cancer immunotherapeutic agent can occur in any order, simultaneously, or they can be administered sequentially, wherein one is given before the other. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

For the treatment of oncological disorders, compounds, agents, and compositions disclosed herein can be administered to a subject in need of treatment prior to, subsequent to, or in combination with other anti-tumor or anti-cancer agents or substances (e.g., chemotherapeutic agents, immunotherapeutic agents, radiotherapeutic agents, cytotoxic agents, etc.) and/or with radiation therapy and/or with surgical treatment to remove a tumor. The administration of the disclosed compositions alone or in combination with an anti-tumor or anti-cancer agents or substances can occur in any order, simultaneously, or they can be administered sequentially, wherein one is given before the other.

### Formulations

In vivo application of the disclosed compositions can be accomplished by any suitable method and technique presently or prospectively known to those skilled in the art. For example, the disclosed compositions can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compositions can be a single administration, or at continuous or distinct intervals as can be readily determined by a person skilled in the art.

The compounds disclosed herein, and compositions comprising them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein, and compositions comprising them, can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds and compositions disclosed herein can be formulated such that an effective amount of the compound is combined with a suitable carrier in order to facilitate effective administration of the composition. The compositions used can also be in a variety of forms. These include, for example, solid, semi-solid, and liquid dosage forms, such as tablets, pills, powders, liquid solutions or suspension, suppositories, injectable and infusible solutions, and sprays. The form depends on the intended mode of administration and therapeutic application. The compositions can also include conventional pharmaceutically-acceptable carriers and diluents which are known to those skilled in the art. Examples of carriers or diluents for use with the compositions include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

In some examples, compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compositions disclosed herein can be systemically administered, such as intravenously or orally, optionally in combination with a pharmaceutically acceptable carrier such as an inert diluent, or an assimilable edible carrier for oral delivery. They can be enclosed in hard- or soft-shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

The compounds disclosed herein, and compositions comprising them, including pharmaceutically acceptable salts or prodrugs thereof, can be administered intravenously, intramuscularly, or intraperitoneally by infusion or injection. Solutions of the active agent or its salts can be prepared in water, optionally mixed with a non-toxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, isotonic agents, for example, sugars, buffers or sodium chloride can be used. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the methods of preparation can be vacuum-drying and the freeze-drying techniques, which yield a powder of the active ingredient plus any additional desired ingredient present in the previously sterile-filtered solutions.

Dosages of the compositions disclosed herein will depend upon the mode of administration, the disease or condition to be treated, and the individual subject's condition, and can be determined in a routine manner. More specifically, the dosage will vary with the age, condition, sex and extent of the disease in the patient, whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. Effective dosages and schedules for administering the compositions can be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like.

### EXAMPLES

The following examples are put forth so as to provide those of ordinary skill in the art with a complete disclosure and description of how the compounds and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Methods. CBD, WFA, and TMZ combinations were screened for synergy in vitro using the human cell lines U-87 and U-251 MG. Single-agent treatments were performed for comparison. Cellular toxicity assays were also done to find the viability of the cells after treatment with individual and combination treatments, along with synergistic calculations for the combinations. Treatments were given to both cell lines for 48 hours. To investigate the mechanisms responsible for synergy, qPCR was used to detect changes in gene targets responsible for apoptosis, inflammation, proliferation, stress response, and cell cycle arrest.

### Example 2

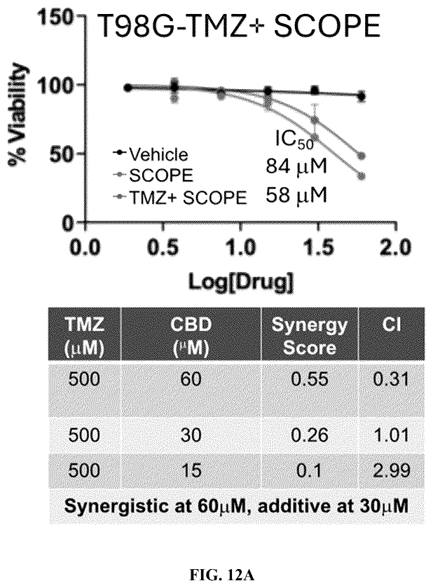

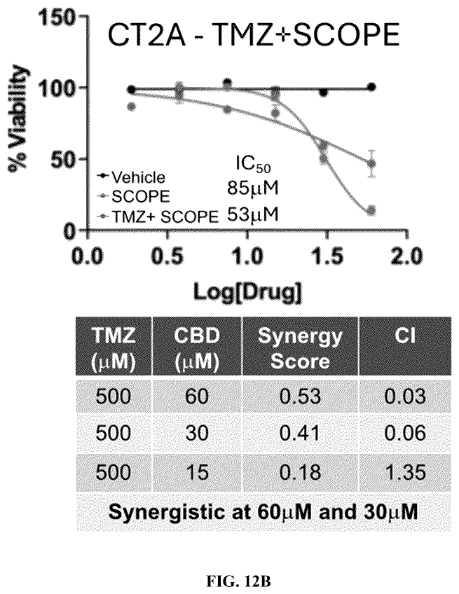

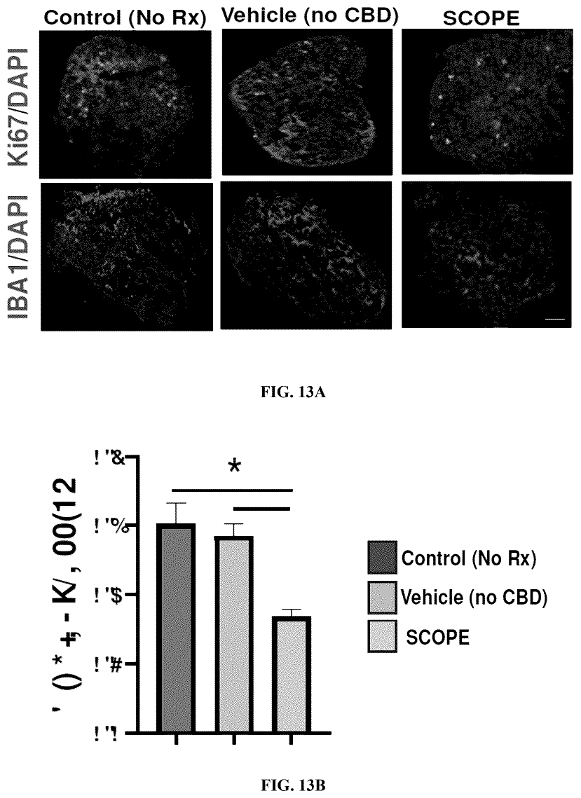

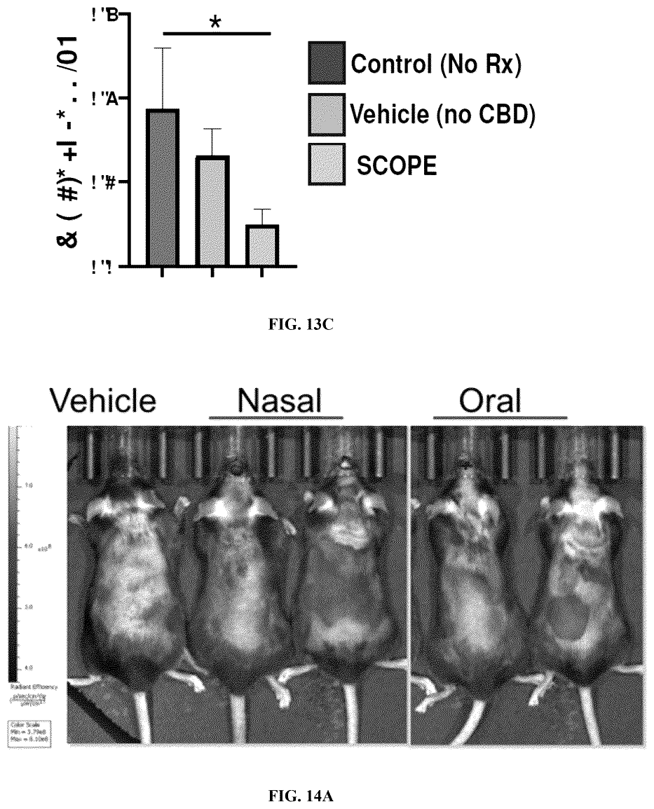

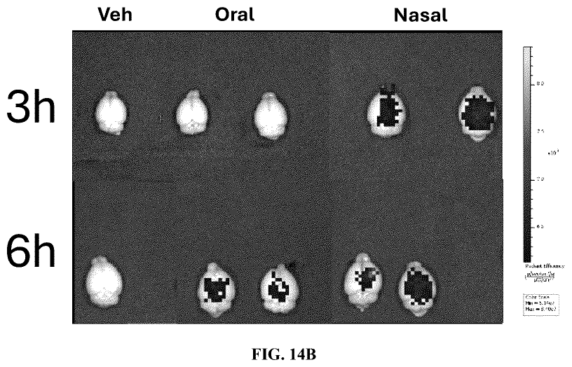

The efficacy of SCOPE vs. control was tested in CT-2A and T98G cells. The results of the viability assay showed that TMZ+SCOPE treatment significantly reduced IC₅₀ of CBD and exhibited either synergy or additive effects at 30-60 μM depending upon cell line (FIGS. 12A-12B). Administration of the control alone did not show any change in viability in the dose ranges tested. In addition, in murine GBM-derived organized cultures, only the treatment with CBD nanoemulsion (SCOPE) showed a significant reduction in Ki67 and IBA1 staining, suggesting a significant decrease in GBM organoid proliferation and inflammation, respectively (FIGS. 13A-13B).

What is claimed is:

1. A composition for treating CBD- and/or TMZ-refractory glioblastoma, comprising a cannabinoid, temozolomide (TMZ), a CCL20/CCR6 inhibitor, or a pharmaceutically acceptable salt thereof.

2. The composition of claim 1, wherein the cannabinoid is CBD or a pharmaceutically acceptable salt thereof.

3. A pharmaceutically acceptable composition or formulation, comprising the composition of claim 1.

4. A method of treating CBD- and/or TMZ-refractory glioblastoma, comprising administering a therapeutically effective amount of a cannabinoid, TMZ, a CCL20/CCR6 inhibitor, or pharmaceutically acceptable salts thereof.

5. The method of claim 4, wherein the cannabinoid comprises cannabinol (CBN), cannabinolic acid (CBNA), tetrahydrocannabinol (THC), tetrahydrocannabinolic acid (THCA), cannabidiol (CBD), cannabinolic acid (CBDA), tetrahydrocannabivarin (THCV), tetrahydrocannabinol acetate ester (THCOA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabidivarin (CBDV), cannabichromevarin (CBCV), cannabigerovarin (CBGV), cannabigerol monomethyl ether (CBGM), β-caryophyllene, β-caryophyllene oxide, pharmaceutically acceptable salts thereof or any combination thereof.

6. The method of claim 4, wherein the cannabinoid is CBD or a pharmaceutically acceptable salt thereof.

7. The method of claim 4, wherein the CCL20/CCR6 inhibitor comprises a dendrimer nanoparticle complexed with at least one short hairpin RNA (shRNA)-encoding DNA plasmid to form a dendriplex.

8. The method of claim 7, wherein the at least one shRNA-encoding DNA plasmid contains at least one shRNA encoding CCL20, CCR6, or a combination thereof.

9. The method of claim 7, wherein the dendrimer nanoparticle comprises polyamidoamine (PAMAM).

10. The method of claim 4, wherein the cannabinoid further forms a nano-emulsion with a hydroxydocosahexaenoic acid (HDHA).

11. The method of claim 10, wherein the HDHA comprises 14-HDHA, 17-HDHA, pharmaceutically acceptable salts thereof, or a combination thereof.

12. The method of claim 4, wherein the therapeutically effective amount of the cannabinoid, TMZ, the CCL20/CCR6 inhibitor, or pharmaceutically acceptable salts thereof are administered in a single pharmaceutically acceptable composition or formulation.

13. The method of claim 12, wherein the cannabinoid further forms a nano-emulsion with a HDHA.

14. The method of claim 13, wherein the HDHA comprises 14-HDHA, 17-HDHA, pharmaceutically acceptable salts thereof, or a combination thereof.

15. The method of claim 4, wherein the therapeutically effective amount of the cannabinoid, TMZ, the CCL20/CCR6 inhibitor, or pharmaceutically acceptable salts thereof are administered in separate pharmaceutically acceptable compositions or formulations.

16. The method of claim 15, wherein the cannabinoid further forms a nano-emulsion with a HDHA.

17. The method of claim 16, wherein the HDHA comprises 14-HDHA, 17-HDHA, pharmaceutically acceptable salts thereof, or a combination thereof.

18. The method of claim 15, wherein the separate pharmaceutically acceptable compositions or formulations are administered sequentially or simultaneously.

19. The method of claim 4, further comprising administering a targeted therapy to a subject in need thereof.

20. The method of claim 19, wherein the targeted therapy comprises immunotherapy, chemotherapy, surgery, radiotherapy, or ionizing radiation.
